(19) United States
(12) Patent Application Publication
(10) Pub. No.: US 2019/0024597 A1
(43) Pub. Date: Jan. 24, 2019

(54) SMOKE AMOUNT ESTIMATION DEVICE AND COMBUSTION SYSTEM CONTROL DEVICE

(71) Applicant: DENSO CORPORATION, Kariya-city, Aichi-pref. (JP)

(72) Inventors: Atsunori OKABAYASHI, Kariya-city (JP); Shinya HOSHI, Kariya-city (JP)

(21) Appl. No.: 15/773,607

(22) PCT Filed: Oct. 18, 2016
(86) PCT No.: PCT/JP2016/080764

§ 371 Date: May 4, 2018

(30) Foreign Application Priority Data
Nov. 12, 2015 (JP) 2015-222317

(51) Int. Cl. F02D 41/14; F02D 45/00; G01N 33/22; F02D 41/00

(52) U.S. Cl. CPC F02D 41/1456; F02D 45/00; G01N 33/22; F02D 35/023; F02D 41/1441; F02D 41/0002; F02D 41/0077

(57) ABSTRACT

A smoke amount estimation device includes a component amount acquisition unit and an estimation unit. The component amount acquisition unit acquires the amount of aromatic components contained in a fuel to be used for the combustion of an internal combustion engine, and acquires the amount of aromatic variable components that are components that decompose and polymerize before combustion to form aromatic components among components contained in the fuel. The estimation unit estimates the amount of smoke contained in the exhaust gas discharged from the internal combustion engine based on the amount of aromatic components and the amount of aromatic variable components acquired by the component amount acquisition unit.

### CROSS REFERENCE TO RELATED APPLICATION

This application is based on Japanese Patent Application No. 2015-222317 filed on Nov. 12, 2015, the disclosure of which is incorporated herein by reference.

### TECHNICAL FIELD

The present disclosure relates to a smoke amount estimation device that estimates an amount of smoke included in exhaust gas of an internal combustion engine, and to a combustion system control device that controls operation of a combustion system.

### BACKGROUND ART

It is conventionally desired to accurately estimate an amount of smoke contained in the exhaust gas of an internal combustion engine. The smoke is formed of particulate components (PM) in the exhaust gas and contains soot as its main component, and the soot is formed with a number of aromatic components polymerized and laminated. Therefore, the smoke amount tends to increase to a larger amount, as a larger amount of aromatic components are contained in a fuel. In view of this point, Patent Document 1 discloses that a smoke amount is estimated based on the amount of the aromatic components contained in a fuel.

However, the present inventors conducted various tests and found that when different fuels are used, smoke amounts may be greatly different from each other, even if the amounts of aromatic components contained in the fuels are equal to each other. That is, according to the conventional method of estimating a smoke amount based on the amount of aromatic components, there is a limit on an improvement in estimation accuracy.

### PRIOR ART DOCUMENT

### Patent Document

PATENT DOCUMENT 1: JP 2007-46477 A

### SUMMARY OF INVENTION

An object of the present disclosure is to provide both a smoke amount estimation device that can estimate a smoke amount with high accuracy and a combustion system control device.

According to one embodiment of the present disclosure, a smoke amount estimation device includes: a component amount acquisition unit that acquires the amount of aromatic components contained in a fuel to be used for the combustion of an internal combustion engine, and acquires the amount of aromatic variable components that are components that decompose and polymerize before combustion to form aromatic components among components contained in the fuel; and an estimation unit that estimates the amount of smoke contained in the exhaust gas discharged from the internal combustion engine based on the amount of aromatic components and the amount of aromatic variable components acquired by the component amount acquisition unit.

According to another embodiment of the present disclosure, a combustion system control device that controls operation of a combustion system having an internal combustion engine includes: a component amount acquisition unit that acquires the amount of aromatic components contained in a fuel to be used for combustion of the internal combustion engine, and acquires the amount of aromatic variable components that are components that decompose and polymerize before combustion to form aromatic components among components contained in the fuel; an estimation unit that estimates the amount of smoke contained in the exhaust gas discharged from the internal combustion engine based on the amount of aromatic components and the amount of aromatic variable components acquired by the component amount acquisition unit; and a control unit that controls the operation of the combustion system based on the smoke amount estimated by the estimation unit.

The molecular structure of a fuel, before being burned after being injected into a combustion chamber, changes due to being exposed to a high temperature environment. One of the changes is that the below-described aromatic variable components decompose by thermal decomposition or radicals and polymerize, whereby they change to aromatic components. Specific examples of the aromatic variable components include naphthenes, paraffins, and the like. Aromas have a cyclic structure having an unsaturated bond, and the aromatic variable components change to have such a structure.

For example, naphthenes have a cyclic structure, but do not have an unsaturated bond. Even such naphthenes may change to aromas as described below. That is, bonds between atoms may be partially broken due to thermal decomposition or the like and further hydrogen may be extracted by a hydrogen abstraction reaction, whereby the broken site may be bonded to another site, and as a result, naphthenes may change to have a cyclic structure having an unsaturated bond, that is, change to aromas. Paraffins do not have a cyclic structure, but they may change to have a cyclic structure having an unsaturated bond, that is, change to aromas by decomposing and polymerizing in the same way.

In the combustion chamber, aromatic components are polymerized and laminated to form soot just before combustion, and most of the soot disappears by combustion. Soot remaining without being burned is discharged from the combustion chamber, which becomes a smoke component contained in the exhaust gas. Therefore, as a larger amount of aromatic components are contained in a fuel, a smoke amount becomes larger.

However, aromatic variable components may change to aromatic components just before combustion, as described above, and hence the amount of aromatic components may be large just before combustion, even for the fuel containing a low amount of aromatic components in a state of normal temperature and normal pressure. This means that even if the amount of aromatic components contained in a fuel is equal, a smoke amount differs when the amount of aromatic variable components differs.

Based on this knowledge, the amount of aromatic variable components is acquired in addition to the amount of aromatic components, so that a smoke amount is estimated based on both the amount of aromatic components and that of aromatic variable components, in the first invention and the second invention. Therefore, a smoke amount is estimated also in consideration of a change in the molecular structure of a fuel, generated before combustion, and hence the smoke amount can be estimated with high accuracy.

### BRIEF DESCRIPTION OF DRAWINGS

The above and other objects, characteristics, and advantages of the present disclosure will become more apparent from the following detailed description with reference to the accompanying drawings:

FIG. 1 is a view for explaining a combustion system control device according to a first embodiment of the disclosure and a combustion system of an internal combustion engine to which the device is applied;

FIG. 2 is a view for explaining an ignition delay time;

FIG. 3 is a view for explaining a relationship among a plurality of ignition delay times, combustion conditions that are a combination of combustion environment values representing flammability, and mixing amounts of various components;

FIG. 4 is a view showing a relationship between a property line representing a change in the ignition delay time caused due to an in-cylinder oxygen concentration and the molecular structure species of fuel;

FIG. 5 is a view showing a relationship between a property line representing a change in the ignition delay time caused due to an in-cylinder temperature and the molecular structure species of fuel;

FIG. 6 is a view showing a relationship between a property line specified based on an ignition delay time and the mixing ratio of a molecular structure species;

FIG. 7 is a flowchart showing a process flow of a microcomputer shown in FIG. 1, which shows procedures for controlling operation of a combustion system;

FIG. 8 is a view for explaining a method of estimation processing in FIG. 7, which explains relationships between the mixing amounts of various components and smoke amounts;

FIG. 9 is a view showing relationships between threshold values to be used in the determination processing in FIG. 7 and smoke amounts;

FIG. 10 is a graph showing a correlation between the smoke amount estimated by the method of FIG. 9 and the actually measured smoke amount; and

FIG. 11 is a functional block view showing, for each block, functions exerted by a microcomputer according to a second embodiment of the present disclosure.

### DESCRIPTION OF EMBODIMENTS

Hereinafter, embodiments of a smoke amount estimation device and a combustion system control device according to the present disclosure will be described with reference to the views. In each embodiment, parts corresponding to the items described in the preceding embodiment are denoted by the same reference numerals, and duplicated description may be omitted. In each embodiment, when only part of a configuration is described, the previously described other embodiments can be referred to and applied to the other parts of the configuration.

### First Embodiment

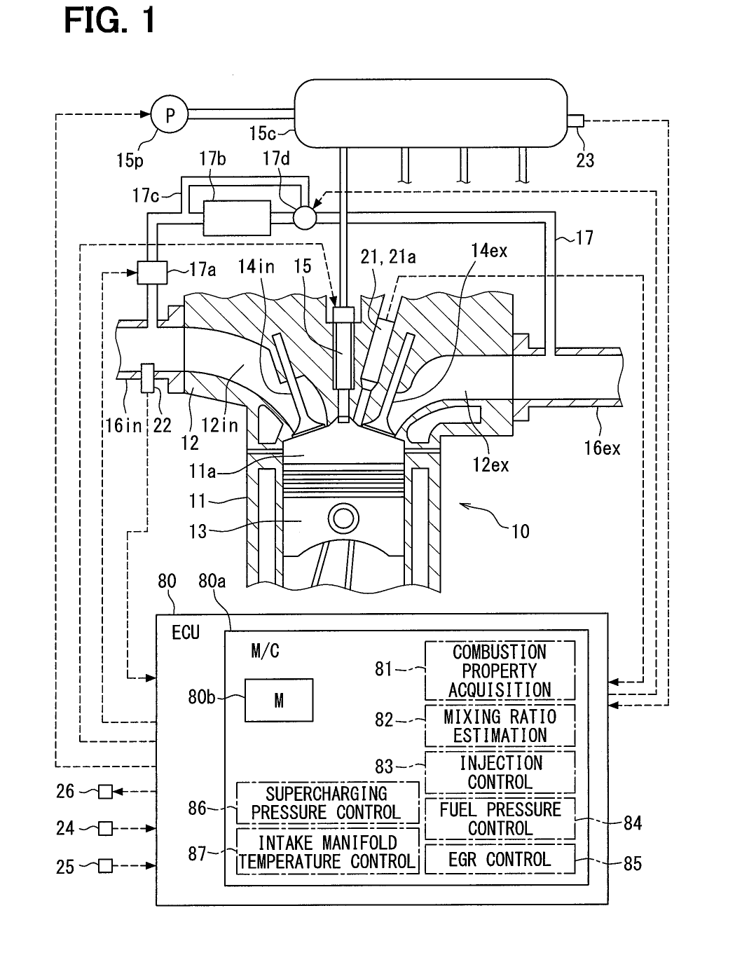

A combustion system control device according to the present embodiment is provided by an electronic control unit (ECU) 80 shown in FIG. 1. The ECU 80 includes a microcomputer 80a, an unshown input processing circuit and an output processing circuit, and the like. The microcomputer 80a includes an unshown central processing unit (CPU) and a memory 80b. With the CPU executing a predetermined program stored in the memory 80b, the microcomputer 80a controls the operations of a fuel injection valve 15, a fuel pump 15p, an EGR valve 17a, a temperature control valve 17d, a supercharging pressure regulator 26, and the like, which are included in a combustion system. Through these controls, the combustion state of an internal combustion engine 10 included in the combustion system is controlled to be a desired state. The combustion system and the ECU 80 are mounted in a vehicle, and the vehicle travels by using the output of the internal combustion engine 10 as a driving source.

An internal combustion engine 10 includes a cylinder block 11, a cylinder head 12, a piston 13, and the like. An intake valve 14in, an exhaust valve 14ex, a fuel injection valve 15, and an in-cylinder pressure sensor 21 are attached to the cylinder head 12.

The fuel pump 15p pumps the fuel in the fuel tank to the common rail 15c. The fuel in the common rail 15c is stored therein in a state in which the pressure of which is maintained at a target pressure Ptrg with the ECU 80 controlling the operation of the fuel pump 15p. The common rail 15c distributes the accumulated fuel to the fuel injection valve 15 of each cylinder. The fuel injected from the fuel injection valve 15 mixes with the intake air in a combustion chamber 11a to form an air-fuel mixture, and the air-fuel mixture is compressed by the piston 13 and self-ignites. The internal combustion engine 10 is a compression self-ignition type diesel engine, and light oil is used as fuel.

The fuel injection valve 15 is configured by accommodating, in the body, an electromagnetic actuator and a valve body. When the ECU 80 powers on the electromagnetic actuator, the electromagnetic attraction force of the electromagnetic actuator opens a leak passage of an unshown back pressure chamber, and the valve body opens with a decrease in back pressure and an injection hole formed in the body is opened, whereby fuel is injected from the injection hole. When the electromagnetic actuator is powered off, the valve body closes, whereby the fuel injection is stopped.

An intake pipe 16in and an exhaust pipe 16ex are respectively connected to an intake port 12in and an exhaust port 12ex formed in the cylinder head 12. An EGR pipe 17 is connected to each of the intake pipe 16in and the exhaust pipe 16ex, so that EGR gas that is part of exhaust gas flows (refluxes) into the intake pipe 16in through the EGR pipe 17. An EGR valve 17a is attached to the EGR pipe 17. The aperture of the EGR pipe 17 is controlled with the ECU 80 controlling the operation of the EGR valve 17a, whereby the flow rate of the EGR gas is controlled.

In addition, an EGR cooler 17b for cooling the EGR gas, a bypass pipe 17c, and a temperature control valve 17d are attached to the upstream portion of the EGR valve 17a of the EGR pipe 17. The bypass pipe 17c forms a bypass flow path through which the EGR gas bypasses the EGR cooler 17b. The temperature control valve 17d adjusts a ratio between the EGR gas flowing through the EGR cooler 17b and the EGR gas flowing through the bypass flow path and finally adjusts the temperature of the EGR gas flowing into the intake pipe 16in by adjusting the aperture of the bypass flow path. The intake air flowing into the intake port 12in contains external air (fresh air) flowing into from the intake pipe 16in and the EGR gas. Therefore, adjusting the temperature of the EGR gas by the temperature control valve 17d corresponds to adjusting an intake manifold temperature that is the temperature of the intake air flowing into the intake port 12in.

The combustion system includes an unshown supercharger. The supercharger has a turbine to be attached to the exhaust pipe 16ex and a compressor to be attached to the intake pipe 16in. When the turbine rotates by the flow velocity energy of the exhaust, the compressor rotates by the rotational force of the turbine, whereby the fresh air is compressed or supercharged by the compressor. The above-described supercharging pressure regulator 26 is a device for changing the capacity of the turbine, and the turbine capacity is adjusted with the ECU 80 controlling the operation of the supercharging pressure regulator 26, whereby the supercharging pressure by the compressor is controlled.

Detection signals detected by various sensors, such as the in-cylinder pressure sensor 21, an oxygen concentration sensor 22, a rail pressure sensor 23, a crank angle sensor 24, and an accelerator pedal sensor 25, are inputted to the ECU 80.

The in-cylinder pressure sensor 21 outputs a detection signal corresponding to the pressure (in-cylinder pressure) of the combustion chamber 11a. The in-cylinder pressure sensor 21 has a temperature detection element 21a in addition to a pressure detection element, and also outputs a detection signal corresponding to the temperature (in-cylinder temperature) of the combustion chamber 11a. The oxygen concentration sensor 22 is attached to the intake pipe 16in, and outputs a detection signal corresponding to the oxygen concentration of the intake air. The intake air to be detected is a mixture of fresh air and the EGR gas. The rail pressure sensor 23 is attached to the common rail 15c, and outputs a detection signal corresponding to the pressure (rail pressure) of the accumulated fuel. The crank angle sensor 24 outputs a detection signal corresponding to the rotation speed of a crankshaft rotationally driven by the piston 13, that is, to the rotation number (engine rotation number) of the crankshaft per unit time. The accelerator pedal sensor 25 outputs a detection signal corresponding to the depression amount (engine load) of an accelerator pedal to be depressed by a vehicle driver.

Based on these detection signals, the ECU 80 controls the operations of the fuel injection valve 15, the fuel pump 15p, the EGR valve 17a, the temperature control valve 17d, and the supercharging pressure regulator 26. Thereby, a fuel injection start timing, an injection amount, an injection pressure, an EGR gas flow rate, an intake manifold temperature, and a supercharging pressure are controlled.

A microcomputer 80, while controlling the operation of the fuel injection valve 15, functions as an injection control unit 83 that controls a fuel injection start timing, an injection amount, and the number of injection stages related to multi-stage injection. The microcomputer 80a, while controlling the operation of a fuel pump 15p, functions as a fuel pressure control unit 84 that controls an injection pressure. The microcomputer 80a, while controlling the operation of an EGR valve 17a, functions as an EGR control unit 85 that controls an EGR gas flow rate. The microcomputer 80a, while controlling the operation of a temperature control valve 17d, functions as an intake manifold temperature control unit 87 that controls an intake manifold temperature. The microcomputer 80a, while controlling the operation of a supercharging pressure regulator 26, functions as a supercharging pressure control unit 86 that controls a supercharging pressure.

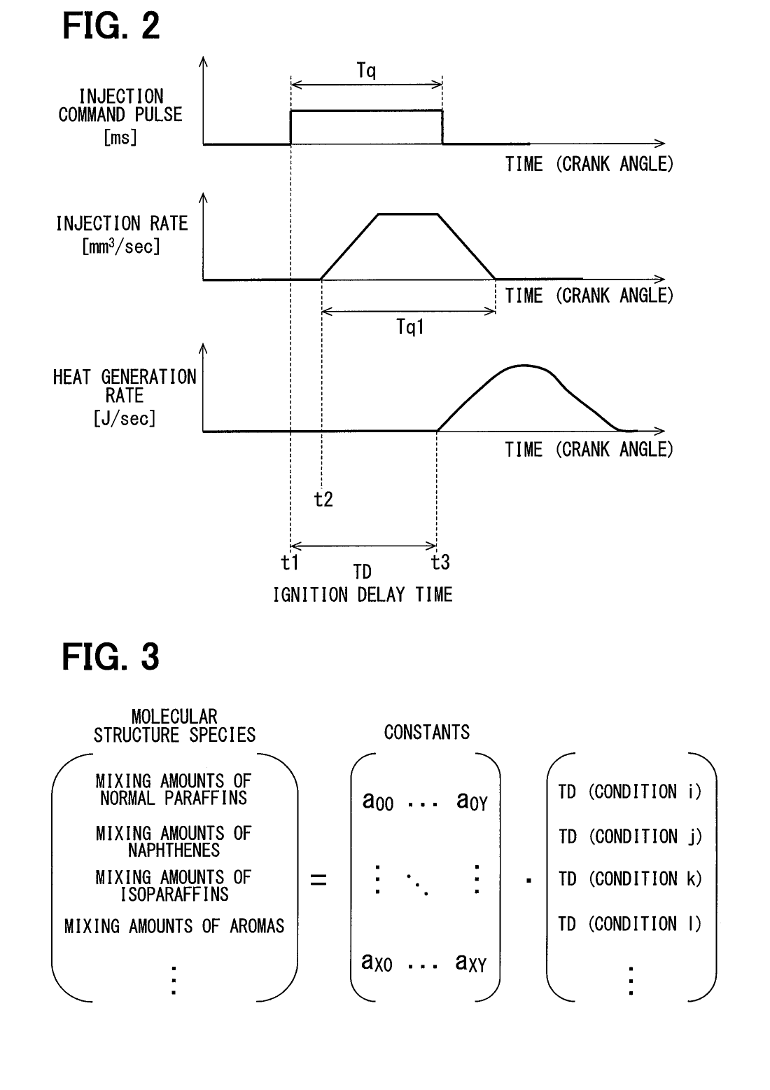

The microcomputer 80a also functions as a combustion property acquisition unit 81 that acquires a detected value (combustion property value) of a physical quantity related to combustion. The combustion property value according to the present embodiment is an ignition delay time TD shown in FIG. 2. The upper graph in FIG. 2 shows a pulse signal outputted from the microcomputer 80a. Powering the fuel injection valve 15 is controlled in accordance with the pulse signal. Specifically, the powering is started at a pulse-on timing t1, and is continued for a pulse-on period Tq. In short, an injection start timing is controlled by a pulse-on timing. In addition, an injection period is controlled by the pulse-on period Tq, which finally controls an injection amount.

The middle graph in FIG. 2 shows a change in the injection state of fuel from the injection hole, the change being generated as a result of the fact that the valve body opens and closes in accordance with the pulse signal. Specifically, a change in the injection amount (injection rate) of fuel injected per unit time is shown. As shown in the graph, there is a time lag between the timing t1 at which the powering is started and a timing t2 at which injection is actually started. There is also a time lag between a timing at which the powering is ended and a timing at which the injection is actually stopped. A period Tq1 for which the injection is actually being performed is controlled by the pulse-on period Tq.

The lower graph in FIG. 2 shows a change in the combustion state of the injected fuel in the combustion chamber 11a. Specifically, a change in a heat amount (heat generation rate) per unit time is shown, the change being caused with a mixture of the injected fuel and the intake air self-igniting and burning. As shown in the graph, there is a time lag between the timing t2 at which the injection is started and a timing t3 at which combustion is actually started. In the present embodiment, the time between the timing t1 at which powering is started and the timing t3 at which combustion is started is defined as the ignition delay time TD.

The combustion property acquisition unit 81 estimates the timing t3 at which the combustion is started based on a change in the in-cylinder pressure detected by the in-cylinder pressure sensor 21. Specifically, a timing at which the in-cylinder pressure suddenly rises during a period for which a crank angle rotates by a predetermined amount after the piston 13 reaches a top dead center, is estimated as a combustion start timing (timing t3). The ignition delay time TD is calculated by the combustion property acquisition unit 81 based on this estimation result. The combustion property acquisition unit 81 further acquires various states (combustion conditions) during combustion for each combustion. Specifically, at least one of an in-cylinder pressure, an in-cylinder temperature, an intake oxygen concentration, an injection pressure, and air-fuel mixture flow velocity is acquired as a combustion environment value.

These combustion environment values are parameters representing the flammability of fuel, and it can be said that each of the in-cylinder pressure just before combustion, the in-cylinder temperature just before combustion, the intake oxygen concentration, the injection pressure, and the air-fuel mixture flow velocity increases to a higher level, the air-fuel mixture is more likely to self-ignite and burn. As the in-cylinder pressure and in-cylinder temperature just before combustion, for example, the values, detected at the timing t1 at which powering the fuel injection valve 15 is started, may be used. The in-cylinder pressure is detected by the in-cylinder pressure sensor 21, the in-cylinder temperature by the temperature detection element 21a, the intake oxygen concentration by the oxygen concentration sensor 22, and the injection pressure by the rail pressure sensor 23. The air-fuel mixture flow velocity is the flow velocity of the air-fuel mixture in the combustion chamber 11a just before combustion. Since this flow velocity becomes higher as the engine rotation number becomes larger, it is calculated based on the engine rotation number. The combustion property acquisition unit 81 stores the acquired ignition delay time TD in the memory 80b in association with a combination (combustion conditions) of the combustion environment values related to the combustion.

The microcomputer 80a also functions as a mixing ratio estimation unit 82 that estimates mixing ratios of various components contained in a fuel based on a plurality of combustion property values detected under different combustion conditions. The mixing amounts of various components are calculated, for example, by substituting the ignition delay times TD for respective different combustion conditions into the determinant shown in FIG. 3. The mixing ratios of various components are calculated by dividing the respective calculated mixing amounts by the total amount.

The matrix on the left side of FIG. 3 is x rows and 1 column, and the numerical values of this matrix represent the mixing amounts of various components. The various components are components classified according to the types of molecular structures. The types of the molecular structures include normal paraffins, isoparaffins, naphthenes, and aromas.

The matrix on the left side of the right side is x rows and y columns, and the numerical values of this matrix represent constants determined based on the experiments carried out beforehand. The matrix on the right side of the right side is y rows and 1 column, and the numerical values of this matrix represent the ignition delay times TD acquired by the combustion property acquisition unit 81. For example, the numerical value of the first row and first column is the ignition delay time TD (condition i) acquired under a combustion condition i including a predetermined combination of the combustion environment values, and the numerical value of the second row and first column is the ignition delay time TD (condition j) acquired under a combustion condition j. Between the combustion conditions i and j, all of the combustion environment values are set to be different from each other. In the following description, an in-cylinder pressure, an in-cylinder temperature, an intake oxygen concentration, and an injection pressure related to the combustion condition i are set to P (condition i), T (condition i), O₂ (condition i), and Pc (condition i), respectively. An in-cylinder pressure, an in-cylinder temperature, an intake oxygen concentration, and an injection pressure related to the combustion condition j are set to P (condition j), T (condition j), O₂ (condition j), and Pc (condition j), respectively.

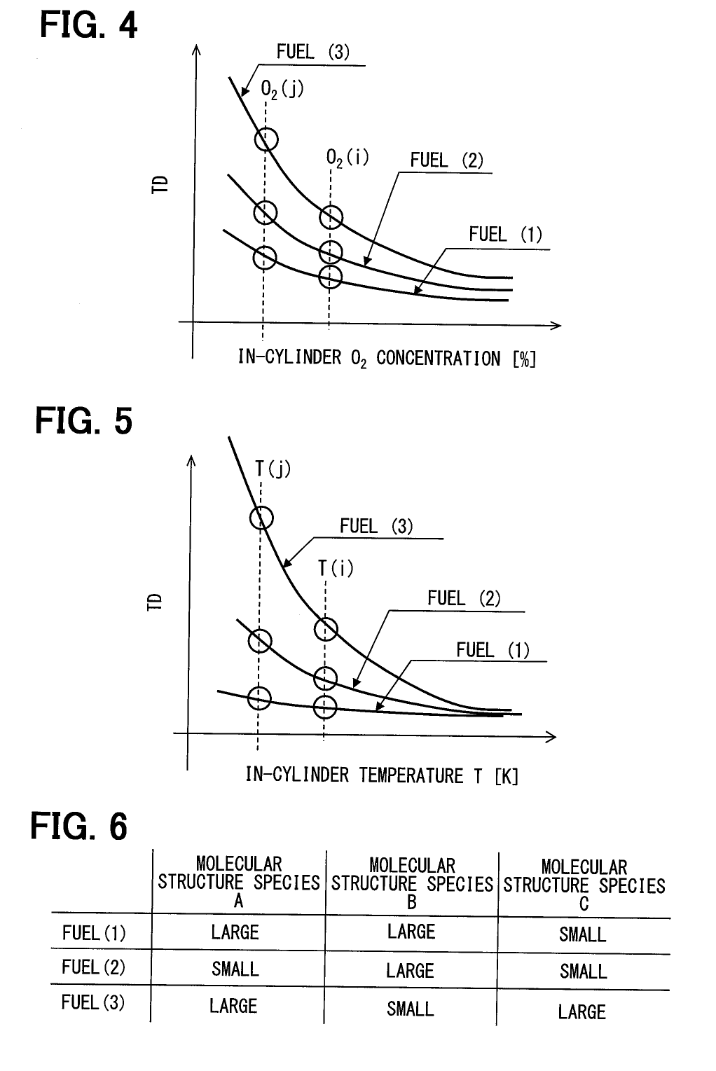

Next, the theory that the mixing ratio of each molecular structure species can be calculated by substituting the ignition delay times TD for respective combustion conditions into the determinant of FIG. 3 will be described with reference to FIGS. 4, 5, and 6.

As the concentration of oxygen (in-cylinder oxygen concentration) contained in an air-fuel mixture related to combustion is higher, the mixture is more likely to self-ignite, and hence the ignition delay time TD becomes shorter, as shown in FIG. 4. Three solid lines (1), (2), and (3) in the view are property lines each showing the relationship between the in-cylinder oxygen concentration and the ignition delay time TD. However, this property line differs depending on fuel. Strictly speaking, the property line differs depending on the mixing ratio of each molecular structure species contained in fuel. Therefore, by detecting the ignition delay time TD occurring when the in-cylinder oxygen concentration is O₂ (condition i), it can be estimated which molecular structure species is contained. In particular, by comparing the ignition delay time TD occurring when the in-cylinder oxygen concentration is O₂ (condition i) with the ignition delay time TD occurring when the in-cylinder oxygen concentration is O₂ (condition j), the mixing ratio can be estimated with higher accuracy.

Similarly, as the in-cylinder temperature is higher, the air-fuel mixture is more likely to self-ignite, and hence the ignition delay time TD becomes shorter, as shown in FIG. 5. Three solid lines (1), (2), and (3) in the view are property lines each showing the relationship between the in-cylinder temperature and the ignition delay time TD. However, this property line differs depending on fuel, and strictly speaking, it differs depending on the mixing ratio of each molecular structure species contained in fuel. Therefore, by detecting the ignition delay time TD occurring when the in-cylinder temperature is B1, it can be estimated which molecular structure species is contained. In particular, by comparing the ignition delay time TD occurring when the in-cylinder temperature is T (condition i) with the ignition delay time TD occurring when the in-cylinder temperature is T (condition j), the mixing ratio can be estimated with higher accuracy.

Similarly, as the injection pressure is higher, oxygen is more likely to be taken in and the air-fuel mixture is more likely to self-ignite, and hence the ignition delay time TD becomes shorter. Strictly speaking, a sensitivity differs depending on the mixing ratio of each molecular structure species contained in fuel. Therefore, by detecting the ignition delay time TD occurring when the injection pressure is different, the mixing ratio can be estimated with higher accuracy.

In addition, a molecular structure species having a high influence on the property line related to the in-cylinder oxygen concentration (see FIG. 4) is different from a molecular structure species having a high influence on the property line related to the in-cylinder temperature (see FIG. 5). Thus, molecular structure species having high influences on the property lines each related to each of a plurality of combustion conditions are different from each other. Therefore, based on a combination of the ignition delay times TD acquired by setting a combination of a plurality of the combustion environment values (combustion conditions) to different values, it can be estimated with high accuracy which molecular structure species is mixed in a large amount, as shown in, for example, FIG. 6. In the following description, the in-cylinder oxygen concentration is referred to as a first combustion environment value, the in-cylinder temperature as a second combustion environment value, and a property line related to the first combustion environment value as a first property line, and a property line related to the second combustion environment value as a second property line.

A molecular structure species A shown in FIG. 6 is one having a high influence on a property line (hereinafter referred to as the first property line) related to the in-cylinder oxygen concentration as the first combustion environment value. A molecular structure species B is one having a high influence on a property line (hereinafter referred to as the second property line) related to the in-cylinder temperature as the second combustion environment value, and a molecular structure species C is one having a high influence on a third property line related to a third combustion environment value. It can be said that as a change in the ignition delay time TD becomes larger with respect to a change in the first combustion environment value, a larger amount of the molecular structure species A is mixed. Similarly, it can be said that as a change in the ignition delay time TD becomes larger with respect to a change in the second combustion environment value, a larger amount of the molecular structure species B is mixed, and it can be said that as a change in the ignition delay time TD becomes larger with respect to a change in the third combustion environment value, a larger amount of the molecular structure species C is mixed. Therefore, the mixing ratios of the molecular structure species A, B, and C can be estimated for each of the different fuels (1), (2), and (3).

Next, the processing of the program executed by the combustion property acquisition unit 81 will be described. This processing is executed each time when the below-described pilot injection is commanded. Injection may be controlled such that a fuel is injected from the same fuel injection valve 15 more than once (multi-stage injection) during one combustion cycle. Of these multiple times of injection, the injection in which the largest injection amount is set is referred to as main injection, and the injection just before that as pilot injection.

First, the combustion property acquisition unit 81 calculates the ignition delay time TD related to the pilot injection by estimating the combustion start timing t3 based on the value detected by the in-cylinder pressure sensor 21, as described above. Next, the ignition delay time TD is stored in a memory 80b in association with a combination of a plurality of the combustion environment values (combustion conditions).

Specifically, a numerical range within which each combustion environment value can fall is divided into a plurality of regions, so that combinations of the regions of a plurality of the combustion environment values are preset. For example, the ignition delay time TD (condition i) shown in FIG. 3 represents the ignition delay time TD acquired when the regions of P (condition i), T (condition i), O₂ (condition i), and Pc (condition i) are combined. Similarly, the ignition delay time TD (condition j) represents the ignition delay time TD acquired when the regions of P (condition j), T (condition j), O₂ (condition j), and Pc (condition j) are combined.

When there is a high possibility that another fuel may have mixed with the fuel stored in the fuel tank when a user has supplied the other fuel, it is assumed that the mixing ratios of molecular structure species have been changed, and the values of the estimated mixing amounts are reset. For example, when an increase in the remaining fuel amount is detected, during the stop of the operation of the internal combustion engine 10, by a sensor that detects the amount of the fuel remaining in the fuel tank, the above values are reset.

The combustion property acquisition unit 81 calculates the mixing amount of each molecular structure species by substituting the ignition delay times TD into the determinant of FIG. 3. The number of columns of a matrix representing constants is changed in accordance with the number of samples, that is, with the number of the rows of the matrix on the right side of the right side of the determinant. Alternatively, regarding the ignition delay times TD that have not been acquired, preset nominal values are substituted into the matrix of the ignition delay time TD. The mixing ratio of each molecular structure species is calculated based on the mixing amount of each molecular structure species thus calculated.

As described above, the microcomputer 80a also functions as the injection control unit 83, the fuel pressure control unit 84, the EGR control unit 85, the supercharging pressure control unit 86, and the intake manifold temperature control unit 87. These control units set target values based on an engine rotation number, an engine load, an engine cooling water temperature, and the like, and performs feedback control such that control objects become the target values. Alternatively, these control units perform open control with contents corresponding to the target values.

The injection control unit 83 controls the injection start timing, the injection amount, and the number of injection stages by setting the pulse signal in FIG. 2 such that the injection start timing, the injection amount, and the number of injection stages become target values (injection control). The number of injection stages means the number of injection related to the above-described multi-stage injection. Specifically, the on-time (powering time) and the pulse on rising timing (powering start timing) of a pulse signal corresponding to the target values are stored in advance on a map. Then, a powering time and a powering start timing, corresponding to the target values, are acquired from the map such that the pulse signal is set.

In addition, an output torque obtained by injection, and emission state values such as a NOx amount and a smoke amount are stored. Then, in setting the target values based on an engine rotation number, an engine load, and the like in the next and subsequent injection, the target values are corrected based on the values stored as described above. In short, feedback control is performed by correcting the target values such that the deviations between the actual output torque and emission state values and the desired output torque and emission state values are made zero.

The fuel pressure control unit 84 controls the operation of a metering valve that controls the flow rate of the fuel sucked into the fuel pump 15p. Specifically, the operation of the metering valve is feedback-controlled based on a deviation between the actual rail pressure detected by the rail pressure sensor 23 and a target pressure Ptrg (target value). As a result, a discharge amount per unit time, the discharge being performed by the fuel pump 15p, is controlled, and the operation of the metering valve is controlled such that the actual rail pressure becomes the target value (fuel pressure control).

The EGR control unit 85 sets the target value of an EGR amount based on an engine rotation number, an engine load, and the like. The EGR amount is controlled by controlling the aperture of the EGR valve 17a based on this target value (EGR control). The supercharging pressure control unit 86 sets the target value of a supercharging pressure based on an engine rotation number, an engine load, and the like. The supercharging pressure is controlled by controlling the operation of the supercharging pressure regulator 26 based on this target value (supercharging pressure control). The intake manifold temperature control unit 87 sets the target value of an intake manifold temperature based on an outside air temperature, an engine rotation number, an engine load, and the like. The intake manifold temperature is controlled by controlling the aperture of the temperature control valve 17d based on this target value (intake manifold temperature control).

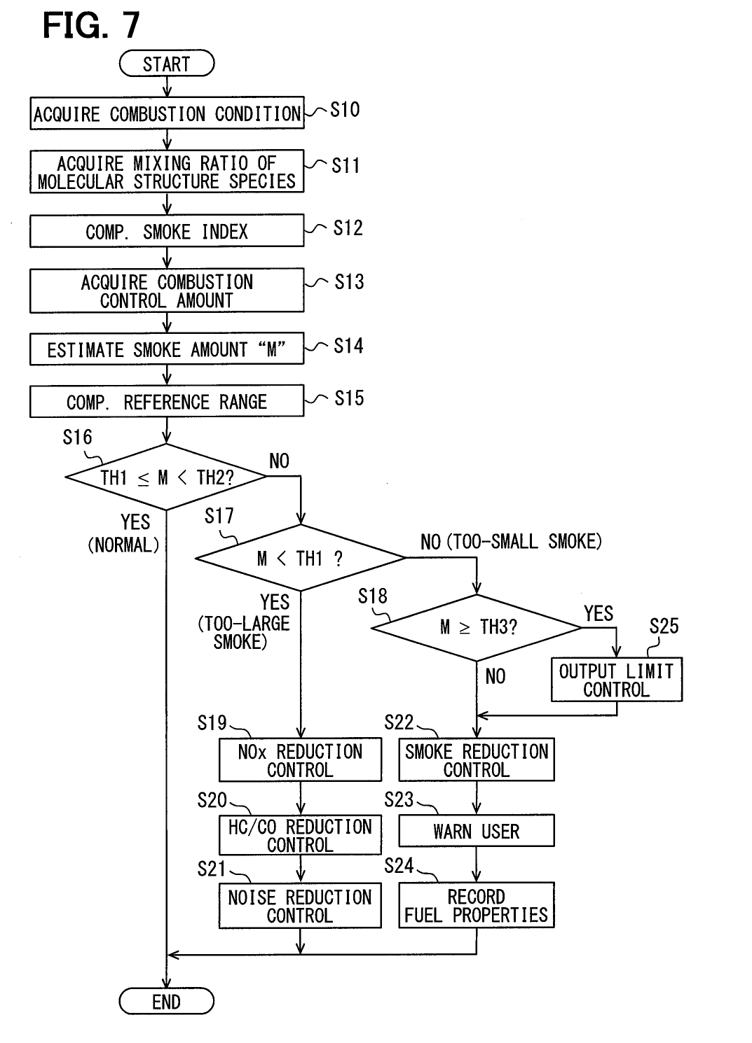

Further, the target values set by the above-described various control units are also corrected by the mixing ratios estimated by the mixing ratio estimation unit 82. Processing procedures for executing this correction by the microcomputer 80a will be described below with reference to FIG. 7. This processing is repeatedly executed at predetermined intervals during the operation period of the internal combustion engine 10.

First, the combustion conditions just before combustion occurs in the combustion chamber 11a, that is, the respective various combustion environment values described above are acquired in Step S10 in FIG. 7. Specifically, at least one of an in-cylinder pressure, an in-cylinder temperature, an intake oxygen concentration, an injection pressure, and an air-fuel mixture flow velocity is acquired as the combustion environment value.

In the following Step S11, the mixing ratio estimated by the mixing ratio estimation unit 82 is acquired. That is, the mixing ratio of each of the molecular structure species shown on the left side of FIG. 3 is acquired. The microcomputer 80a, while executing the processing of Step S11, corresponds to the “component amount acquisition unit.” In the following Step S12, a smoke index, which is an index representing how likely smoke is to be generated, is estimated based on the mixing ratio acquired in Step S11.

For each of different fuels with different mixing ratios of various components contained in the fuels, how likely smoke is to be generated (degree of generation) differs, even if the fuel has similar fuel properties such as cetane number and dynamic viscosity. In the present embodiment, an index representing a degree of smoke generation is referred to as a smoke index, and as the value of the smoke index is larger, the degree of smoke generation becomes larger. Among the various components, there are components that greatly influence the smoke index and components that do not significantly influence it. In view of such a degree of influence, the smoke index is calculated based on the mixing ratios of various components. Herein, the smoke index related to a fuel in which each of the various components is mixed at a reference mixing ratio is referred to as a reference smoke index.

As described above, the main component of the smoke contained in the exhaust gas is soot, and the soot is formed with a large number of aromatic components polymerized and laminated. This polymerization reaction occurs with a fuel containing aromatic components exposed to a high temperature environment. Therefore, the soot is generated from the fuel injected into the combustion chamber 11a just before combustion. However, almost all of the generated soot is burned in the combustion chamber 11a just after being formed and disappears. The soot remaining without being burned is discharged from the combustion chamber 11a. The soot thus discharged is the main component of exhaust smoke. To be precise, the above smoke index represents how likely the soot, existing in the combustion chamber 11a just before combustion, is to increase. As a fuel has a higher smoke index, the amount of soot existing just before combustion is larger, and hence the amount of soot remaining without being burned, that is, a smoke amount M becomes larger.

As described above, soot is generated from the fuel injected into the combustion chamber 11a just before combustion. Therefore, as the mixing ratio of aromatic components, among the mixing ratios of the respective molecular structure species acquired in Step S11, is larger, the smoke index becomes higher. In addition, the above-described aromatic variable components can be changed to aromatic components just before combustion, and hence as the mixing ratio of aromatic variable components, among the mixing ratios of the respective molecular structure species acquired in Step S11, is larger, the smoke index becomes higher.

In view of these knowledges, the smoke index is estimated to be a larger value in Step S12, as the mixing ratios of aromatic components and aromatic variable components are larger. In detail, a weighting coefficient representing the degree of influence of aromatic components on the smoke index is set to be larger than that representing the degree of influence of aromatic variable components on the smoke index.

Among the aromatic variable components, for an aromatic variable component that is more likely to change to an aromatic component, a weighting coefficient is set to be larger. Specific examples of the aromatic variable components include, for example, naphthene components, isoparaffin components, normal paraffin components, and the like. Since naphthene components, isoparaffin component, and normal paraffin components are less likely to change to aromatic components in this order, the weighting coefficients are set to be smaller in this order.

Among naphthene components, naphthene components each having a structure having two or more of cyclic structures are more likely to change to aromatic components. Therefore, the weighting coefficient for naphthene components each having a structure having two or more of cyclic structures is set to be larger than that for naphthene components each having a structure having less than two of cyclic structures.

Among isoparaffin components, isoparaffin components, each having a structure having carbon atoms whose number is smaller than the average number of carbon atoms of a plurality of types of components contained in a fuel, are more likely to change to aromatic components. Therefore, the weighting coefficient for isoparaffin components each having a structure having carbon atoms whose number is smaller than the average number of carbon atoms is set to be larger than that for isoparaffin components each having a structure having carbon atoms whose number is equal to or larger than the average number of carbon atoms.

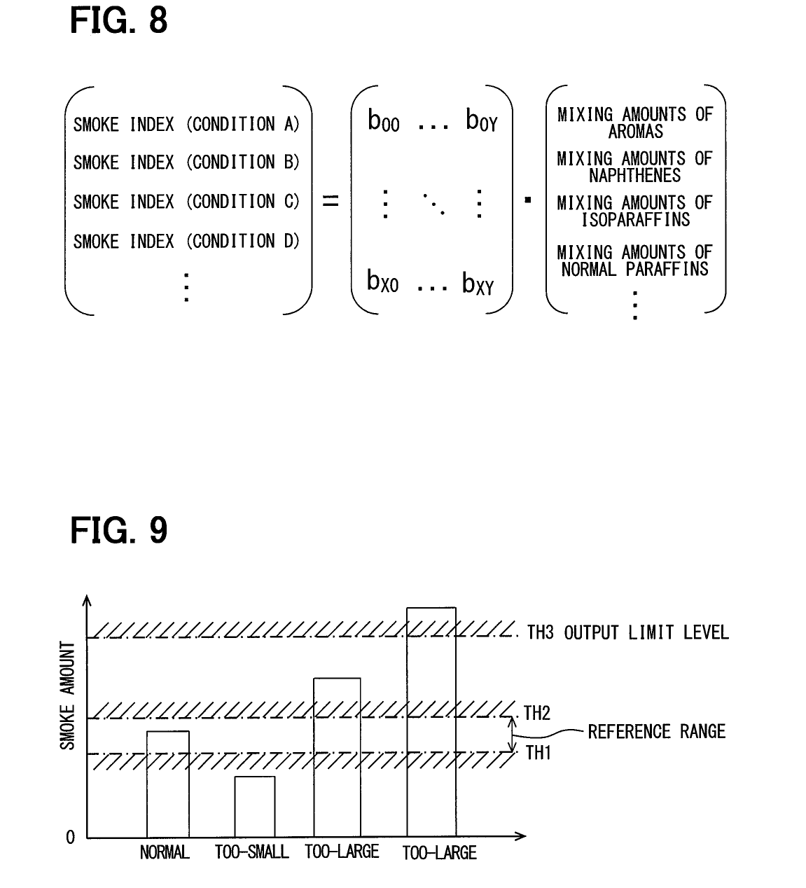

For example, a smoke index for each of combustion conditions A, B, C, and D is calculated by substituting the mixing ratio of each molecular structure species into the determinant shown in FIG. 8. The matrix on the left side of FIG. 8 is x rows and 1 column, and each of the numerical values of this matrix represents the smoke index for each of the different combustion conditions A, B, C, and D. Each of the combustion conditions A, B, C, and D is specified by a combination of a plurality of combustion environment values. Specific examples of the combustion environment values include an in-cylinder pressure, an in-cylinder temperature, an intake oxygen concentration, an injection pressure, an air-fuel mixture flow velocity, and the like. Each of the combustion conditions A, B, C, and D is specified, for example, by dividing each combustion environment value into a plurality of regions and by combining the different regions of the respective combustion environment values.

The matrix on the left side of the right side of FIG. 8 is x rows and y columns, and the numerical values of this matrix represent constants determined based on the tests conducted in advance. The matrix on the right side of the right side is y rows and 1 column, and the numerical values of this matrix represent the estimated values of the mixing amounts of components classified according to the types of molecular structures, the estimated values being calculated by the method of FIG. 3, or the like.

The types of molecular structures related to the substitution into the determinant of FIG. 8 include both aromatic variable components such as normal paraffins, isoparaffins, and naphthenes and aromas. The mixing amounts of the naphthene components are calculated with respect to naphthenes each having a structure having two or more of cyclic structures and naphthenes each having a structure having less than two of cyclic structures. Among the naphthene components, naphthene components each having a structure having two or more of cyclic structures are more likely to change particularly to aromatic components. Therefore, a weighting coefficient for naphthene components each having a structure having two or more of cyclic structures is set to be larger than that for naphthene components each having a structure having less than two of cyclic structures. Herein, naphthenes each having a structure having less than two of cyclic structures are less likely to change to aromas than naphthenes each having a structure having two or more of cyclic structures, and hence substitution of them into the determinant may be omitted.

The mixing amounts of the isoparaffin components are calculated with respect to isoparaffins each having a structure having a small number of carbon atoms and isoparaffins having a structure having a large number of carbon atoms. Specifically, the above classification is made by calculating an average number of carbon atoms of a plurality of types of components contained in the fuel and based on whether the number of carbon atoms of the relevant isoparaffins is smaller than the average number of carbon atoms. Among isoparaffin components, side chain paraffin components, each having a structure having carbon atoms whose number is smaller than the average number of carbon atoms of a plurality of types of components contained in the fuel, are more likely to change particularly to aromatic components. Therefore, the weighting coefficient for isoparaffin components each having a structure having carbon atoms whose number is less than the average number of carbon atoms is set to be larger than that for isoparaffin components each having a structure having carbon atoms whose number is equal to or larger than the average number of carbon atoms. Herein, isoparaffins each having a structure having a large number of carbon atoms are less likely to change to aromas than isoparaffins each having a structure having a small number of carbon atoms, and hence substitution of them into the determinant may be omitted.

In the following Step S13, respective control amounts by the injection control unit 83, the fuel pressure control unit 84, the EGR control unit 85, the supercharging pressure control unit 86, and the intake manifold temperature control unit 87 are acquired as combustion control amounts. The specific control amounts by the injection control unit 83 include, for example, a fuel injection amount and a fuel injection timing. In particular, a pilot injection amount greatly influences the smoke amount M.

In the following Step S14, the smoke amount M is estimated based on the combustion environment value acquired in Step S10, the smoke indices calculated in Step S12, and the control amounts acquired in Step S13. The microcomputer 80a, while executing the processing of Step S14, corresponds to the “estimation unit.”

Herein, even if the properties of a fuel, such as dynamic viscosity and cetane number, are the same, the smoke index differs when the mixing ratio of each molecular structure species differs. Therefore, the smoke index is calculated based on the mixing ratio of each molecular structure species in the present embodiment.

Even if the mixing ratio of each molecular structure species is equal, the smoke index differs depending on the combustion environment value. As the combustion environment value is one at which combustion is more likely to occur, the amount of soot to be burned and disappear becomes larger, and hence the amount of soot remaining without being burned, that is, the smoke amount M is reduced to a smaller amount. For example, as an ignition delay time between when a fuel is injected and when the fuel ignites is longer, the mixing property of the fuel with air is improved to a higher level, and hence the amount of soot to be burned and disappear becomes larger and the smoke amount M is reduced to a smaller amount. For example, as the environment in the combustion chamber 11a, occurring just before combustion, is higher in oxygen concentration, higher in flow velocity, and higher in temperature, the amount of soot to be burned and disappear is larger and the smoke amount M is reduced to a smaller amount. Therefore, the smoke index is set, in Step S14, in accordance with a combination of the plurality of types of combustion environment values (combustion condition) that have been acquired. Specifically, smoke indices suitable for the acquired combustion condition are selected from the plurality of smoke indices shown on the left side of FIG. 8.

As the smoke index is higher, the smoke amount M is estimated to be larger. However, even if the smoke index is equal, the smoke amount M differs when the combustion control amounts differ. For example, assuming that as the amount of heat generated as a result of combustion, which is estimated based on the combustion control amounts, is larger, the amount of soot to be burned and disappear becomes larger, the smoke amount M is estimated to be smaller. Also, assuming that as the ignition delay time TD, estimated based on the combustion control amounts, is longer, the amount of soot to be burned and disappear becomes larger because the mixing property of the fuel with air is improved to a higher level, the smoke amount M is estimated to be smaller. As described above, the smoke amount M is estimated, in Step S14, based on both the smoke index suitable for a combustion condition and the combustion control amounts.

In Step S14, the smoke amount M may be calculated by calculating a smoke index by substituting the mixing ratio of each molecular structure species into a first arithmetic expression and then by substituting the smoke index, the combustion environment values, and the combustion control amounts into a second arithmetic expression. Alternatively, the smoke amount M may be calculated by substituting the mixing ratio of each molecular structure species, the combustion environment values, and the combustion control amounts into a third arithmetic expression without calculating a smoke index. These arithmetic expressions may be stored in advance in the microcomputer 80a or the like.

In the following Step S15, a reference range of the smoke amount is calculated based on an appropriate range of the smoke index stored in advance, the combustion environment value acquired in Step S10, and the control amounts acquired in Step S13. This reference range is a range of the smoke amount assumed when a proper fuel is used. For example, a numerical range of the reference smoke index corresponding to the combustion environment values is mapped in association with the combustion environment values and stored in advance, and the numerical range of the smoke index suitable for the combustion environment value acquired in Step S10 is acquired by referring to the map. A lower limit value TH1 of the reference range of the smoke amount is calculated from both the lower limit value of the numerical range of the acquired smoke index and the control amounts. In addition, an upper limit value TH2 of the reference range of the smoke amount is calculated from both the upper limit value of the smoke index and the control amounts. Thereby, the reference range of the smoke amount is calculated.

In the following Step S16, it is determined whether the smoke amount M estimated in Step S14 is within the reference range calculated in Step S15. When it is determined that it is out of the reference range, it is determined, in the following Step S17, which one of a smoke too-small state, in which the smoke amount M is less than the lower limit value TH1, and a smoke too-large state, in which the smoke amount M is equal to or larger than the upper limit value TH2, occurs. Specifically, it is determined whether the smoke amount M is less than the lower limit value TH1.

When it is determined that the smoke too-large state occurs, it is determined in the following Step S18 whether the smoke amount M is equal to or larger than a limit value TH3. The limit value TH3 is set to a value larger than the upper limit value TH2, and is calculated, for example, by adding a predetermined amount to the upper limit value TH2 calculated in Step S15 or by multiplying the upper limit value TH2 by a predetermined coefficient.

In short, the microcomputer 80a, while executing the processing of Steps S16 and S17, corresponds to a “determination unit.” The determination unit determines which one of a normal state, in which the smoke amount estimated in Step S14 (estimation unit) is within the reference range, a too-large state, in which the smoke amount is large beyond the reference range, and a too-small state, in which the smoke amount is smaller than the reference range, occurs. FIG. 9 shows the relationships between each of the reference range and the limit value TH3 and each of the normal state, the too-large state, and the too-small state.

When it is determined in Step S16 that the smoke amount M is within the reference range, it is assumed that a proper fuel is used and the processing in FIG. 7 is ended. As a result, when a proper fuel is used, the above-described control (normal control) by the injection control unit 83, the fuel pressure control unit 84, the EGR control unit 85, the supercharging pressure control unit 86, and the intake manifold temperature control unit 87 are executed.

There is a trade-off relationship between those of the amounts of NOx, HC, and CO that are contained in exhaust gas and the magnitude of combustion noise, and the amount of smoke generated. Therefore, when it is determined in Step S17 that the smoke too-small state occurs, various control amounts by the normal control are corrected in the following Steps S19, S20, and S21, so that the NOx amount, the HC amount, the CO amount, and the combustion noise are reduced instead of increasing the smoke amount.

For example, the actual EGR amount is reduced by lowering the target value of the EGR amount related to the EGR control unit 85 in Step S19. Alternatively, the actual intake manifold temperature is lowered by lowering the target value of the intake manifold temperature related to the intake manifold temperature control unit 87. Thereby, the NOx amount is reduced. In Step S20, various control amounts are corrected to reduce the HC amount and the CO amount. In Step S21, various control amounts are corrected to reduce combustion noise.

On the other hand, when it is determined in Step S17 that the smoke too-large state occurs, and when it is determined in Step S18 that the smoke amount M is less than the limit value TH3, the processing proceeds to the following Step S22. In Step S22, various control amounts by the normal control are corrected to reduce the smoke amount, instead of increasing the NOx amount, the HC amount, the CO amount, and the combustion noise.

In the following Step S23, a user is warned that an improper fuel in which the smoke too-large state occurs is used. In the following Step S24, the properties of the improper fuel currently in use are recorded. For example, the mixing ratio of a molecular structure species, acquired in Step S11, is stored in the memory 80b. When it is determined in Step S18 that the smoke amount M is equal to or larger than the limit value TH3, various control amounts are changed in the following Step S25, so that the output by the internal combustion engine 10 is limited to one less than a predetermined value. The microcomputer 80a, while executing the processing of Steps S19, S20, S21, S22, and S25, corresponds to the “control unit.”

In the present embodiment, the component amount acquisition unit is included as described above, the component amount acquisition unit acquiring both the amount of aromatic components contained in the fuel and the amount of aromatic variable components that are components that decompose and polymerize before combustion to form aromatic components. Further, the estimation unit of Step S14 is included, the estimation unit estimating the smoke amount M based on the amount of aromatic components and the amount of aromatic variable components acquired by the component amount acquisition unit. Therefore, the smoke amount M is estimated in consideration of the amount of aromatic components that are the sources of soot and also in consideration of the amount of aromatic variable components whose molecular structures change to aromatic components before combustion, and hence the smoke amount M can be estimated with high accuracy. Herein, the decomposition includes thermal decomposition and decomposition by radicals, and strictly speaking, decomposition by radicals occurs after thermal decomposition occurs.

Further, at least naphthene components are contained in the aromatic variable components whose amount is to be acquired by the component amount acquisition unit in the present embodiment. Among various aromatic variable components, naphthene components are particularly likely to change to aromatic components. Therefore, according to the embodiment in which the amount of naphthene components is included in the amount of aromatic variable components that is used for the estimation of the smoke amount, the accuracy of estimating the smoke amount M can be improved.

Furthermore, in the present embodiment, at least naphthene components, each having a structure having two or more of cyclic structures, are contained in the naphthene components whose amount is to be acquired by the component amount acquisition unit. Among naphthene components, naphthene components, each having a structure having two or more of cyclic structures, are particularly likely to change to aromatic components. Therefore, according to the embodiment in which the amount of naphthene components, each having a structure having two or more of cyclic structures, is included in the amount of aromatic variable components to be used for the estimation of the smoke amount, the accuracy of estimating the smoke amount M can be improved.

Still furthermore, in the present embodiment, at least isoparaffin components are contained in the aromatic variable components whose amount is to be acquired by the component amount acquisition unit. Among various aromatic variable components, naphthene components are particularly likely to change to aromatic components. Therefore, according to the embodiment in which the amount of isoparaffin components is included in the amount of aromatic variable components to be used for the estimation of the smoke amount, the accuracy of estimating the smoke amount M can be improved.

Still furthermore, in the present embodiment, at least isoparaffin components, each having a structure having carbon atoms whose number is smaller than the average number of carbon atoms of a plurality of types of components contained in the fuel, are contained in the isoparaffin components whose amount is to be acquired by the component amount acquisition unit. Among the isoparaffin components, isoparaffin components, each having a structure having a small number of carbon atoms, are particularly likely to change to aromatic components. Therefore, according to the embodiment in which the amount of isoparaffin components, each having a structure having carbon atoms whose number is smaller than the average number of carbon atoms, is included in the amount of aromatic variable components to be used for the estimation of the smoke amount, the accuracy of estimating the smoke amount M can be improved.

Still furthermore, in the present embodiment, the amount of smoke corresponding to the combustion environment values, such as the temperature, pressure, oxygen concentration, and the like of the combustion chamber 11a, is estimated based on the amount of aromatic components and the amount of aromatic variable components. Specifically, the smoke index for the respective combustion environment values is calculated based on the mixing ratio of each molecular structure species contained in the fuel. Then, a smoke index corresponding to the actual combustion environment values is selected from the calculated smoke indices, which is used for the estimation of the smoke amount M. Therefore, the accuracy of estimating the smoke amount M can be improved.

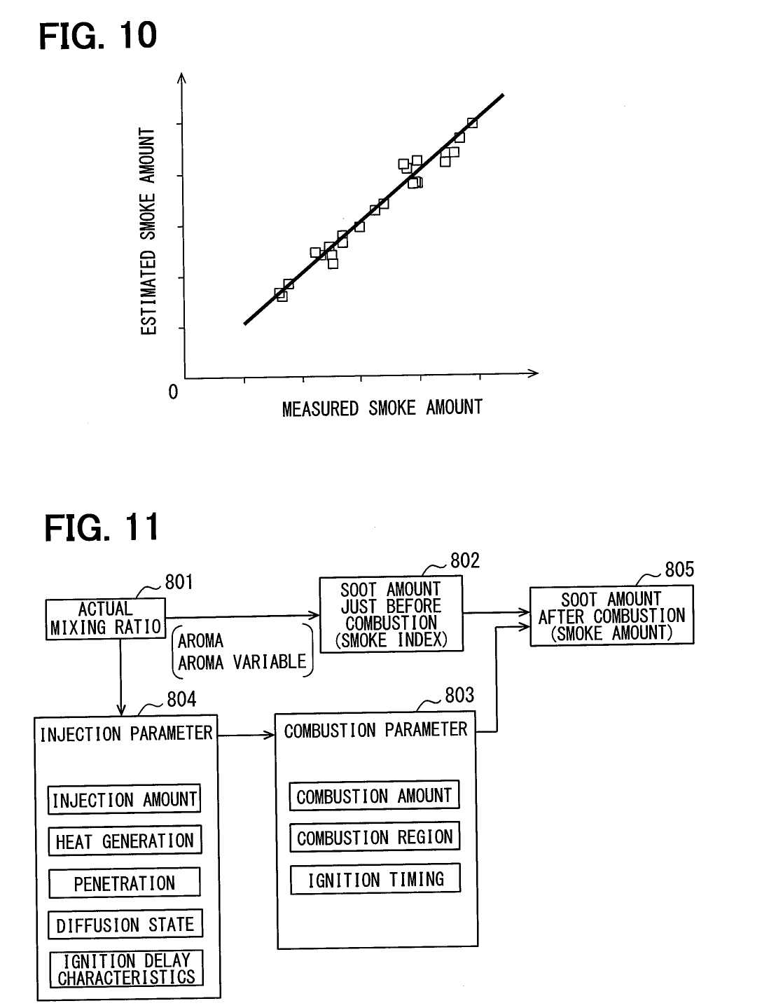

This effect is confirmed by the present inventors, as shown in the below-described test result of FIG. 10. In this test, the amount of smoke discharged per unit time is measured for each of different combustion environment value and each time when a different fuel is burned. In addition, at least the amounts of aromatic components and aromatic variable components are acquired for each of the combustion environment values and the fuels used in the test. Then, the smoke amount M is estimated based on the acquired component amounts and the combustion environment values by the above-described method. The horizontal axis in FIG. 10 represents the measurement results of the smoke amount, and the vertical axis represents the estimation results of the smoke amount M. It is confirmed from this test result that deviations between the estimated values and the measured values are small for all combustion environment values and fuels and sufficient estimation accuracy is obtained.

Still furthermore, in the present embodiment, the above-described component amount acquisition unit and the estimation unit are included, and a control unit that controls the operation of the combustion system based on the smoke amount estimated by the estimation unit is included. Specific examples of the control unit include the injection control unit 83, the fuel pressure control unit 84, the EGR control unit 85, the supercharging pressure control unit 86, and the intake manifold temperature control unit 87.

Herein, when the mixing ratio of each of the various components contained in a fuel differs, an optimal content for operating the combustion system in a desired state differs, even if a fuel having the same fuel properties (e.g., cetane number) is used. Examples of the plurality of types of components include, for example, a component (smoke factor component) that has a large influence on the amount of smoke generated, a component that has a large influence on the amount of NOx generated, and a component that has a large influence on the amount of heat generated.

In the present embodiment in which this point is taken into consideration, the smoke amount M is estimated based on the mixing ratio of the amount of aromatic components and the mixing ratio of the amount of aromatic variable components, both the components being smoke factor components, and the injection control, fuel pressure control, EGR control, supercharging pressure control, intake manifold temperature control, and the like are controlled based on the estimated value. Therefore, control for obtaining a desired smoke amount M can be achieved with higher accuracy than conventional control in accordance with the properties of a fuel, such as cetane number. In particular, the balance among various states, such as the smoke amount M, HC amount, CO amount, combustion noise, output torque, and fuel consumption rate, can be controlled to a desired state with high accuracy.

Still furthermore, the combustion property acquisition unit 81 and the mixing ratio estimation unit 82 are included in the present embodiment. The combustion property acquisition unit 81 acquires a detected value of a physical quantity related to the combustion of the internal combustion engine 10 as a combustion property value. The mixing ratio estimation unit 82 estimates the mixing ratios of various components contained in the fuel based on a plurality of combustion property values detected under different combustion conditions.

Herein, even if exactly the same fuel is burned, combustion property values, such as an ignition delay time and the amount of heat generated, differ when the combustion conditions at the time, such as an in-cylinder pressure and an in-cylinder temperature, differ. For example, in the case of the fuel (1) in FIG. 4, the ignition delay time TD (combustion property value) becomes shorter as the combustion is performed under a condition in which the in-cylinder oxygen concentration is higher. The degree of change in the combustion property value with respect to a change in the combustion condition, that is, the property lines shown by the solid lines in FIG. 4 differ for each of the fuels (1), (2), and (3) in each of which the mixing ratio of each molecular structure species is different from the other two. In the present embodiment in which this point is taken into consideration, the mixing ratio of each molecular structure species contained in the fuel is estimated based on a plurality of the ignition delay times TD (combustion property values) detected under different combustion conditions, whereby the properties of a fuel can be accurately grasped.

Still furthermore, in the present embodiment, the combustion condition is one specified by a combination of a plurality of types of combustion environment values. That is, for each of the plurality of types of combustion environment values, a combustion property value, occurring when combustion is performed under a condition in which a combustion environment value is different, is acquired. According to this, a mixing ratio can be estimated with higher accuracy than in the case where for the same type of combustion environment values, a combustion property value, occurring when combustion is performed under a condition in which the combustion environment values are different, is acquired such that a mixing ratio is estimated based on the combustion condition and the combustion property values.

Still furthermore, in the present embodiment, at least one of the in-cylinder pressure, the in-cylinder temperature, the intake oxygen concentration, and the fuel injection pressure is included in the plurality of types of combustion environment values related to the combustion conditions. According to the embodiment in which a mixing ratio is estimated by using combustion property values occurring when combustion is performed under a condition in which these combustion environment values are different, the mixing ratio can be estimated with high accuracy because these combustion environment values have a large influence on a combustion state.

Still furthermore, in the present embodiment, the combustion property value is the ignition delay time TD between when fuel injection is commanded and when the fuel self-ignites. According to the embodiment in which a mixing ratio is estimated based on the ignition delay time TD, the mixing ratio can be estimated with high accuracy because the ignition delay time TD is greatly influenced by the mixing ratios of various components.

Still furthermore, in the present embodiment, the combustion property acquisition unit 81 acquires a combustion property value related to the combustion of the fuel injected before the main injection (pilot injection). When the fuel of the main injection is burned, the in-cylinder temperature becomes high, and hence the fuel after the main injection is more likely to be burned. Therefore, a change in the combustion property value, occurring due to a difference between the mixing ratios in fuels, is less likely to appear. On the other hand, the fuel injected before the main injection (pilot injection) is not influenced by the main combustion, and hence a change in the combustion property value, occurring due to a difference between the mixing ratios in fuels, is more likely to appear. Therefore, in estimating a mixing ratio based on the combustion property values, the estimation accuracy can be improved.

### Second Embodiment

In the first embodiment, the smoke index is calculated based on the mixing ratio of the amount of aromatic components and the mixing ratio of the amount of aromatic variable components. On the other hand, in the present embodiment, paying attention to the fact that: a combustion state differs depending on the mixing ratio of each molecular structure species; and when a combustion state differs, the amount of soot remaining without being burned differs and a smoke amount differs, a smoke amount is calculated also in view of a combustion state. Specific examples of the combustion state include a combustion amount, a combustion region, an ignition timing, and the like.

As shown in FIG. 11, an acquisition unit 801 acquires the mixing ratio of each molecular structure species estimated by the mixing ratio estimation unit 82 in FIG. 1. A smoke index calculation unit 802 calculates a smoke index based on the mixing ratios of aromatic components and aromatic variable components among the respective mixing ratios that have been acquired. The smoke index is one representing how likely soot is to be generated just before combustion, and as soot is more likely to be generated, the smoke index becomes higher. As described above, as the amounts of aromatic components and aromatic variable components are larger, the amount of soot just before combustion becomes larger and the smoke index becomes higher.

A parameter correlating with the injection amount of the fuel, a parameter correlating with the amount of heat generated, a parameter correlating with a penetration, a parameter correlating with a diffusion state, and a parameter correlating with ignitability are referred to as injection parameters. For example, even if the pressure of the fuel to be supplied to the fuel injection valve 15 and the valve opening time of the fuel injection valve 15 are equal, the injection amounts differ from each other when different fuels are used. Indices, representing an injection amount, an amount of heat generated, a penetration, a diffusion state, and ignitability, these being different from fuel to fuel as described above, are the injection parameters. The penetration means a distance that the fuel, injected from the fuel injection valve 15 to the combustion chamber 11a, reaches in a predetermined time.

These injection parameters have a high correlation with the mixing ratio of each molecular structure species contained in the fuel. Therefore, an injection parameter estimation unit 804 estimates the injection parameter based on the mixing ratio of each of the plurality of types of molecular structure species acquired by the acquisition unit 801. For example, the relationship between the mixing ratio of each molecular structure species and the injection parameter is acquired in advance by conducting tests, so that the injection parameter is calculated from the acquired mixing ratio by using a map or an arithmetic expression representing the above relationship.

A parameter correlating with the combustion amount of the fuel, a parameter correlating with the combustion region, and a parameter correlating with the ignition timing are referred to as combustion parameters. For example, even if conditions, such as the injection amount and the injection timing, are equal, the combustion amounts differ from each other when different fuels are used.

Indices, representing the degrees of change in the combustion amount, the combustion region, and the ignition timing, these being different from fuel to fuel as described above, are the combustion parameter.

These combustion parameters have high correlations with the injection parameters. Therefore, a combustion parameter estimation unit 803 estimates the combustion parameter based on the injection parameter estimated by the injection parameter estimation unit 804. For example, the relationships between the plurality of types of the injection parameters and the respective combustion parameters are acquired in advance by conducting tests, so that each combustion parameter is estimated from the plurality of types of the injection parameters that have been acquired by using a map or an arithmetic expression representing the relationships.

A smoke amount estimation unit 805 calculates the amount of soot (smoke amount) after combustion based on the combustion parameter estimated by the combustion parameter estimation unit 803 and the smoke index estimated by the smoke index calculation unit 802.

According to the present embodiment in which the above configuration is provided, the injection parameter is estimated based on the mixing ratio of each molecular structure species, whereby the injection parameter can be estimated with high accuracy. Then, the combustion parameter is estimated based on the injection parameter thus estimated with high accuracy, whereby the combustion parameter can be estimated with high accuracy. Then, the smoke amount is calculated from the smoke index in consideration of the combustion parameter thus estimated with high accuracy, whereby the smoke amount can be estimated with high accuracy. Therefore, the accuracy of estimating the smoke amount M can be improved according to the embodiment.

### Third Embodiment

In the first embodiment, the mixing ratio estimation unit 82 estimates the mixing ratios of various components based on a plurality of the combustion property values. In the present embodiment, however, the general properties of a fuel are detected by sensors (property sensors), so that the mixing ratios are estimated based on the detection results.

Specific examples of the property sensors include a fuel density sensor, a dynamic viscosity sensor, and the like. The fuel density sensor detects the density of a fuel based on, for example, a natural vibration period measuring method. The dynamic viscosity sensor is, for example, a thin tube viscometer or a dynamic viscometer based on a thin wire heating method, and it detects the dynamic viscosity of the fuel in the fuel tank. The fuel density sensor and the dynamic viscosity sensor include a heater, and detect the density and the dynamic viscosity of the fuel, respectively, in a state in which the fuel is heated to a predetermined temperature by the heater.

The present inventors have paid attention to the fact that: a specific property parameter of a fuel correlates with the physical quantity of each molecular structure contained in a fuel composition; and a sensitivity to the molecular structure differs for each property parameter type. In other words, when a molecular structure differs in a fuel, bonding force between molecules, steric hindrance due to structure, interaction, and the like differ. In addition, a fuel contains a plurality of types of molecular structures, and the mixing amounts thereof differ from fuel to fuel. In this case, it is considered that a sensitivity contributing to a property parameter differs for each molecular structure, and hence the value of a property parameter changes depending on the amount of a molecular structure.

Therefore, the present inventors have established a correlation equation for the property parameters and the molecular structures. This correlation equation is an arithmetic expression of a property calculation model by which a plurality of property parameters are derived by using sensitivity coefficients indicating degrees of dependence of the amounts of a plurality of molecular structures on a plurality of the property parameters and by reflecting the sensitivity coefficients on the amounts of the molecular structures. The amount of a molecular structure contained in the fuel composition can be calculated by inputting, as the values of the property parameters, the values detected by the property sensors to the correlation equation.

In addition, a lower calorific value correlates with the dynamic viscosity and density of a fuel, and hence it can be calculated based on the dynamic viscosity and the density by using a map or an arithmetic expression representing the correlation. The lower calorific value thus calculated may be used as a property parameter to be inputted to the correlation equation.

In addition, the ratio (HC ratio) of the amount of hydrogen to the amount of carbon, which are contained in a fuel, correlates with a lower calorific value, and hence a HC ratio can be calculated based on the lower calorific value by using a map or an arithmetic expression representing the correlation. The HC ratio thus calculated may be used as a property parameter to be inputted to the correlation equation. Other than these, a parameter related to cetane number or distillation property can also be used as the property parameter.

According to the present embodiment, a plurality of property parameters indicating the properties of the fuel are acquired as described above. Then, the amounts of a plurality of molecular structures, that is, the mixing ratio of each molecular structure species is estimated by using correlation data defining correlations between a plurality of property parameters and the amounts of a plurality of molecular structures in the fuel and based on the acquired values of the plurality of property parameters that have been acquired. Therefore, the amounts of aromatic components and aromatic variable components to be used for the estimation of the smoke amount M can be acquired by using the values detected by the property sensors, without using the values detected by the in-cylinder pressure sensor 21.

### Other Embodiments

Although the preferred embodiments of the invention have been described above, the invention is not limited to the above-described embodiments at all, and various modifications can be made as exemplified below. Not only combinations of parts that clearly indicate that combinations are specifically possible in each embodiment, but also partial combinations of the embodiments are possible when there is no particular obstruction to the combinations, even if not explicitly stated.

In the second embodiment, the injection parameter estimation unit 804 and the combustion parameter estimation unit 803 are included, but the injection parameter estimating unit 804 may be excluded and the combustion parameter estimation unit 803 may estimate a combustion parameter based on the mixing ratio of each molecular structure species.

In the embodiment shown in FIG. 2, the time between the timing t1 at which powering is started and the timing t3 at which combustion is started is defined as the ignition delay time TD. On the other hand, the time between the timing t2 at which injection is started and the timing t3 at which combustion is started may be defined as the ignition delay time TD. The timing t2 at which injection is started may be estimated by detecting a timing, at which a change in the fuel pressure such as the rail pressure occurs with the start of injection and based on the detected timing.

The combustion property acquisition unit 81 shown in FIG. 1 acquires the ignition delay time TD as the detected value (combustion property value) of a physical quantity related to combustion. On the other hand, the combustion property acquisition unit 81 may acquire, as the combustion property values, a waveform representing a change in the heat generation rate, an amount of heat (amount of heat generated) generated by the combustion of a corresponding fuel, and the like. In addition, the mixing ratios of various components may be estimated based on a plurality of types of combustion property values such as the ignition delay time TD, the waveform of heat generation rate, and the amount of heat generated. For example, the matrix (constants) on the left side of the right side in FIG. 3 are set to values corresponding to the plurality of types of combustion property values, and the plurality of types of combustion property values are substituted into the matrix on the right side of the right side in FIG. 3, whereby mixing ratios are estimated.

In the example of FIG. 3, the combustion conditions are set such that all of the combustion environment values are different for each of the plurality of the ignition delay times TD. That is, for the respective combustion conditions i, j, k, and l (see FIG. 3) each formed of a predetermined combination of the combustion environment values, all of the in-cylinder pressures are set to different values P (condition i), P (condition j), P (condition k), and P (condition l). Similarly, all of the in-cylinder temperatures T, all of the intake oxygen concentrations O₂, and all of the injection pressures Pc are set to different values. On the other hand, for the respective different combustion conditions, at least one of the combustion environment values may be different. For example, for the respective combustion conditions i and j, all of the in-cylinder temperatures T, all of the intake oxygen concentrations O₂, and all of the injection pressures Pc are set to the same value, and only the in-cylinder pressures may be set to different values P (condition i) and P (condition j).

In the above-described embodiments, combustion property values related to the combustion of the fuel injected just before the main injection (pilot injection) are acquired. On the other hand, combustion property values related to the combustion of the fuel injected after the main injection may be acquired. Specific examples of the injection after the main injection include after-injection and post-injection. When multi-stage injection, in which injection is performed more than once before the main injection, is performed, it is preferable to acquire combustion property values related to the combustion of the fuel injected for the first time, because the combustion is not greatly influenced by the main combustion.

In the above-described embodiments, combustion property values are acquired based on the values detected by the in-cylinder pressure sensor 21. On the other hand, in a configuration not including the in-cylinder pressure sensor 21, combustion property values may be estimated based on the rotational fluctuation (differential value of the rotation number) of a rotation angle sensor. For example, the timing, at which the differential value exceeds a predetermined threshold value due to the pilot combustion, can be estimated as a pilot ignition timing. In addition, a pilot combustion amount can be estimated from the magnitude of the differential value.

In the embodiment shown in FIG. 1, the in-cylinder temperature is detected by the temperature detection element 21a, but the in-cylinder temperature may be estimated based on the in-cylinder pressure detected by the in-cylinder pressure sensor 21. Specifically, the in-cylinder temperature is estimated from the calculation using the in-cylinder pressure, the cylinder volume, the gas weight in the cylinder, and the gas constant.

Means and/or functions provided by the ECU 80 (combustion system control device) can be provided by software recorded on a substantive storage medium, computer executing the software, software only, hardware only, or a combination thereof. For example, when the combustion system control device is provided by a circuit that is hardware, it can be provided by a digital circuit or an analog circuit including many logic circuits.

Although the present disclosure has been described in accordance with embodiments, it is understood that the disclosure should not be limited to the embodiments and structures. The present disclosure encompasses various modifications and variations within the equivalent scope. In addition, various combinations and forms, as well as other combinations and forms including, in them, only one element, more than one, or less, are also within the scope and idea of the disclosure.

1. A smoke amount estimation device comprising:
a component amount acquisition unit that acquires an amount of aromatic components contained in a fuel to be used for combustion of an internal combustion engine, and acquires an amount of aromatic variable components that are components that decompose and polymerize before combustion to form aromatic components among components contained in the fuel; and
an estimation unit that estimates an amount of smoke contained in exhaust gas discharged from the internal combustion engine based on the amount of aromatic components and the amount of aromatic variable components acquired by the component amount acquisition unit.

2. The smoke amount estimation device according to claim 1, wherein
at least naphthene components are contained in the aromatic variable components whose amount is to be acquired by the component amount acquisition unit.

3. The smoke amount estimation device according to claim 2, wherein
at least naphthene components, each having a structure having two or more of cyclic structures, are contained in the naphthene components whose amount is to be acquired by the component amount acquisition unit.

4. The smoke amount estimation device according to claim 1, wherein
at least isoparaffin components are contained in the aromatic variable components whose amount is to be acquired by the component amount acquisition unit.

5. The smoke amount estimation device according to claim 4, wherein
at least isoparaffin components, each having a structure having carbon atoms whose number is smaller than an average number of carbon atoms of a plurality of types of components contained in the fuel, are contained in the isoparaffin
components whose amount is to be acquired by the component amount acquisition unit.

6. The smoke amount estimation device according to claim 1, wherein
at least one of a temperature, a pressure, and an oxygen concentration of a combustion chamber of the internal combustion engine is set as a combustion environment value, and
the estimation unit estimates an amount of smoke corresponding to the combustion environment value based on the amount of aromatic components and the amount of aromatic variable components.

7. The smoke amount estimation device according to claim 1, wherein
parameters, correlating with each of a combustion amount of the fuel, a combustion region, and an ignition timing, are referred to as combustion parameters, and the smoke amount estimation device comprises a combustion parameter estimation unit that estimates at least one of the respective combustion parameters based on a mixing ratio of each molecular structure species contained in the fuel, and
the estimation unit estimates the amount of smoke based on the combustion parameter in addition to the amount of aromatic components and the amount of aromatic variable components.

8. The smoke amount estimation device according to claim 7, wherein
parameters, correlating with each of an injection amount, an amount of heat generated, a penetration, a diffusion state, and ignitability of the fuel injected into the combustion chamber of the internal combustion engine are referred to as injection parameters, and the smoke amount estimation device comprises an injection parameter estimation unit that estimates at least one of the respective injection parameters based on the mixing ratio of each molecular structure species contained in the fuel, and
the combustion parameter estimation unit estimates the combustion parameter by using the injection parameter.

9. A combustion system control device that controls operation of a combustion system including an internal combustion engine, the combustion system control device comprising:
a component amount acquisition unit that acquires an amount of aromatic components contained in a fuel to be used for combustion of the internal combustion engine, and acquires an amount of aromatic variable components that are components that decompose and polymerize before combustion to form aromatic components among components contained in the fuel;
an estimation unit that estimates an amount of smoke contained in exhaust gas discharged from the internal combustion engine based on the amount of aromatic components and the amount of aromatic variable components acquired by the component amount acquisition unit; and
a control unit that controls the operation of the combustion system based on the smoke amount estimated by the estimation unit.

10. The combustion system control device according to claim 9, further comprising
a determination unit that determines which one of a normal state that is a reference range, a too-large state that is larger than the reference range, and a too-small state that is smaller than the reference range the smoke amount estimated by the estimation unit is in, wherein
when the smoke amount is determined to be in the too-large state, the control unit controls to increase at least one of combustion noise and amounts of NOx, HC, and CO in exhaust gas and to reduce the smoke amount, and
when the smoke amount is determined to be in the too-small state, the control unit controls to reduce at least one of combustion noise and the amounts of NOx, HC, and CO in the exhaust gas and to increase the smoke amount.
